(19) United States
(12) Patent Application Publication
(10) Pub. No.: US 2011/0308516 A1
(43) Pub. Date: Dec. 22, 2011

(54) Delivery device for a powder aerosol

(75) Inventors: Robert Price, Chepstow (GB); John Nicholas Staniforth, Bath (GB); Derek Alan Woodcock, Berkhampstead (GB); Paul Michael Young, Sydney (AU)

(73) Assignee: Britannia Pharmaceuticals Ltd., Redhill (GB)

(21) Appl. No.: 12/929,589

(22) Filed: Feb. 2, 2011

Related U.S. Application Data
(63) Continuation of application No. 10/559,372, filed on Nov. 28, 2006, now abandoned.

(30) Foreign Application Priority Data
Jun. 12, 2003 (GB) 0313604
Jun. 14, 2004 (GB) PCT/GB2004/002490

(51) Int. Cl. A61M 11/00

(52) U.S. Cl. 128/200.23

(57) ABSTRACT

The invention provides a delivery device (10) for a medicament comprising:
a housing (50),
a receptacle (54) holding a medicament (80) in the form of a powder; and
a source of propellant (53),
   characterized in that the housing (50) provides an inlet (63) and an outlet (56) for the receptacle (54) wherein the inlet (63) is in fluid communication with the source of propellant (53) and is directed against the medicament (80) and the outlet (56) is spaced from the medicament (80) to allow aerosolisation of the medicament (80); the device (10) provides improved delivery efficiency, particularly a delivered fine particle fraction of greater than 20% by weight.

The present invention is concerned with a hand-held delivery device for a medicament in the form of a powder, typically as an aerosol of powder particles. In particular, the delivery device may be used for delivery of a medicament without a carrier into the airways/lungs.

Two main types of hand held devices for delivering doses of aerosol medicament to a patient are known. These are a propellant-driven metered dose inhaler (MDI) and a dry powder inhaler (DPI).

In an MDI, the medicament is suspended or dissolved in a propellant. The propellant is provided in a pressurised canister having a metered valve which, upon activation, produces a single dose of the medicament in the form of a gas stream. The device may include a tapered discharge nozzle baffle or a venturi to accelerate particles through a discharge nozzle, and to remove oversized particles. Suitable halocarbons used in an MDI include hydrofluorocarbons, hydrofluorochlorocarbons and fluorochlorocarbons having a low boiling point, such as those marketed under the trade mark “Freon”.

The problem with the MDI device is that when it is used to deliver a medicament to a patient's lungs, only a small percentage of the medicament is delivered in a respirable form (approximately 8 weight % fine particle fraction). This is because the high linear speed at which the dosage leaves the device combined with incomplete evaporation of the propellant causes much of the medicament to impact and stick to the back of the throat, causing localised problems in the impact area. This medicament is generally later swallowed by the patient which, for some medicaments such as bronchodilators, can lead to unwanted systemic side effects.

A further problem is that MDIs require coordination between activation and inhalation. Many patients are incapable of this, especially infants, small children and the elderly.

In an attempt to overcome this problem, MDIs have been used with a “spacer” which provides an additional volume in which the propellant may evaporate. It has been found that the fine particle fraction is deposited within the spacer instead of the back of the patient's throat.

In a DPI device, no propellant is used but instead the device relies upon a burst of inspired air drawn through the unit by the patient. These devices suffer from the problem that the force of inspiration varies considerably from person to person. Some patients, particular those with lung problems whom such devices are designed to treat, are unable to generate sufficient air in-flow to activate the device. DPIs have many of the disadvantages of MDIs because of incomplete particle dispersion and the impact at the back of the throat.

In an attempt to overcome this problem with DPIs, the medicament for use in such devices has been formulated in a particular way to aid de-agglomeration. Thus the medicament is generally provided with a carrier or is processed in such a way that weakly bound agglomerations of the medicament are produced which the device may more easily break up. Therefore DPIs are unsuitable for use with medicaments which, due to their high dosage rate, cannot be administered with a carrier or which cannot be further processed in this way. Formulated DPIs where the medicament is administered with a carrier have a problem that the amount of administered medicament in a respirable form is low because the medicament remains adhered to the carrier.

There are other medicaments such as pumactant which is a blend of dipalmitoylphosphatidylcholine (DPPC) and phosphatidylglycerol (PG) (DPPC:PG 7:3), which is very cohesive due to its low particle size, high moisture affinity and predominantly amorphous structure. A device suitable for administering this medicament to the lungs of a patient is needed.

A way of ameliorating these problems has been sought.

In one aspect the present invention provides a delivery device for a powdered medicament comprising:
a housing,
a receptacle holding a medicament in the form of a powder, and
a source of propellant,
   characterized in that the housing has an inlet for the receptacle in fluid communication with the source of propellant and an outlet for medicament wherein the inlet is directed against the medicament and the outlet is spaced from the medicament to allow aerosolisation of the medicament.

A surprising advantage of the device according to the present invention is that it has much greater efficiency than known inhaler devices. It has been found that the device efficiency is about 70.1 weight % in terms of the weight of the delivered dose compared to the weight of the dose loaded in the device (as measured using a Marple Miller impactor; the data is shown in Example 2 below). In particular, the delivered fine particle fraction is at least 20 weight % of the amount of medicament originally loaded in the receptacle. Where the device has been optimised, a delivered fine particle fraction of 40 weight % has been achieved.

The advantages of the spaced arrangement of the outlet (which is the feature that the outlet is spaced from the medicament to allow aerosolisation of the medicament) include that it overcomes the problems of incomplete evaporation of the propellant (where the propellant is liquefied gas) and patient coordination. The problem with patient coordination is improved because there is a slight delay between activation of the device and delivery of the aerosolised medicament from the outlet for the device according to the invention particularly compared to a standard MDI. This is because the aerosol is first generated in the receptacle and then has to pass through the outlet before reaching a patient. This is advantageous because a patient normally finds it difficult to simultaneously activate an inhaler and inhale; it is easier to activate the inhaler and then inhale which the device according to the present invention would allow.

The inlet is generally in fluid communication with the source of propellant such that there is a propellant pathway from the source to the inlet. The propellant pathway is preferably provided with at least one choke to decelerate the propellant. The propellant pathway choke may be in the form of a constriction or a baffle; preferably it is in the form of a constriction. A propellant pathway choke is useful where the medicament is at least partially amorphous such that it is vulnerable to becoming waxy or being compressed when the propellant is directed against it. This would clearly be disadvantageous because an aerosol of the medicament would be generated less efficiently, if at all.

The propellant pathway generally passes from the source of propellant through the housing and then through the header unit to the inlet. It is optionally either formed by the housing or is in the form of tubing, especially medical grade tubing.

The inlet is preferably in the form of an inlet tube. The inlet tube is in fluid communication with the propellant pathway and leads from the housing and is directed against the medicament. The inlet preferably has an end which is directed against the medicament. The end of the inlet is preferably in the form of a flared tube or of a ‘shower-head’ such as a flared and perforated end. The inlet tube preferably extends into the receptacle.

Where it is said that the inlet is directed against the medicament, it should be understood that the inlet is either adjacent to the medicament such that there is a gap between the inlet and the medicament or the inlet is in contact with the medicament. Where the inlet is in contact with the medicament, it is optionally either touching the medicament or inserted into the medicament.

In addition to or as an alternative to a propellant pathway choke, the inlet, particularly the inlet tube, is preferably provided with one or more perforations. Such a perforation is useful as an alternative to a propellant pathway choke as it would decelerate the propellant exiting the inlet before it is directed against the medicament. Furthermore, a perforation in the inlet may also be useful in assisting in the formation of the aerosol of medicament.

In a preferred form of the device according to the invention, the spaced arrangement of the outlet and/or the propellant pathway choke (if present) are preferably arranged such that on activation of the device, a stable aerosol of the medicament is formed in the spaced arrangement. Such a stable aerosol of the medicament will be referred to herein as a standing cloud of medicament.

A device arranged to produce a standing cloud of medicament is particularly advantageous because it makes the medicament easier to administer. Such a device preferably has a normally sealed outlet. Preferably the outlet has an outlet pathway which connects to the exterior of the device (the outlet is in fluid communication with the outlet pathway); more preferably the outlet pathway ends in an exterior outlet; most preferably, the exterior outlet is normally sealed. Such an arrangement is advantageous in terms of patient compliance because a patient is then able to first activate the device to generate the standing cloud of medicament and then open the normally sealed outlet (especially the normally sealed exterior outlet) to inhale the medicament thus avoiding any problem with coordinating activation with inhalation.

The receptacle generally has a bottom containing the medicament and a top which connects to the housing. The outlet is preferably arranged to open into the receptacle at the top of the receptacle. Preferably the outlet is formed as a hole in the housing which is in fluid communication with an outlet pathway to the exterior of the housing.

The source of propellant may optionally be provided by a canister of gas (e.g. compressed gas or liquefied gas) or by a supply of compressed gas such as a supply line of compressed gas such as that typically provided in a hospital room.

The device of the invention is preferably a handheld device using a canister of a pressurized gas as the source of propellant.

The device according to the invention is optionally provided with a mouthpiece attached to the outlet to aid self-administration of the medicament by a patient. Any known mouthpiece may be used in association with the device according to the invention.

Alternatively, the outlet may be provided with a tube for engaging with a breathing tube for a patient using a respirator to enable a third party, e.g. a healthcare professional such as a doctor or nurse to administer a medicament to the patient.

The device has been shown (in Examples 1 and 2) to be highly effective for aerosolizing even highly cohesive powders, such as pumactant. As a result of the high energy transfer, the device also provides a high respirable fraction in the delivered powder and a high delivered dose relative to the loaded dose. Accordingly it provides a vehicle for dispensing powders that hitherto have required formulation with large quantities of excipients, such as lactose, for aerosolisation. This causes problems of bulk when high doses of active are needed. The present invention thus allows active materials that require high doses to be delivered in respirable “drug only” form i.e. without a carrier.

The outlet of the header unit is generally in fluid communication with the exterior of the housing and may be in the form of a passage formed in the header unit or in the form of tubing, especially medical grade tubing. The outlet is preferably provided with one or more chokes for decelerating the aerosol of the medicament where the device is not a device arranged to produce a standing cloud of medicament. Having one or more outlet chokes is useful because it increases the delay between activation of the device and delivery of the medicament, aiding patient compliance. It is also useful because it reduces the problems of reduction in delivered respirable dose because of impact at the back of a patient's throat.

The one or more outlet chokes are preferably one or more constrictions and/or one or more baffles in the outlet. A constriction for use as a choke in the present invention is preferably a reduction in the cross-section of the propellant pathway and/or of the outlet. The reduction in cross-section is optionally either temporary such that after the choke, the propellant pathway and/or outlet revert to their previous cross-section or it is permanent. A baffle for use as a choke in the present invention is preferably provided as an abrupt change in direction of the propellant pathway and/or of the outlet such as a change of direction of from 45 to 135 degrees (measured as the angle between the outlet or propellant pathway before and after the baffle), especially a change of direction of about 90 degrees.

Accordingly, in a further aspect the present invention provides a method of dispensing a medicament as an aerosol to a patient in need of such treatment which method comprises the steps of:
providing a receptacle having an opening which receptacle contains the medicament in powder form;
discharging a pressurised propellant from a canister or cartridge through a delivery tube extending into the receptacle and directed at the medicament so as to fluidise it;
forming an aerosol by transfer of energy from the propellant to the powder; and
discharging the aerosol through an outlet passage provided at the opening of the receptacle.

Where the source of propellant is a removable gas canister and the receptacle is removable, the device may be provided in the form of a first kit according to the invention which kit comprises a gas canister, a receptacle containing a medicament in powder form and a first delivery device housing including the header unit.

Therefore according to the invention, there is provided a first delivery device housing suitable for use in a first kit according to the invention having a first and a second open-ended compartment wherein the first compartment is adapted to receive a source of propellant and the second compartment is adapted to receive a receptacle containing a medicament in powder form wherein the second compartment provides an inlet for propellant in fluid communication with the first compartment and an outlet wherein the outlet, in use, is spaced from the medicament to allow aerosolisation of the medicament.

The first kit optionally further comprises a closure (such as an end cap) for sealing the receptacle in the second compartment.

Alternatively, the receptacle may be provided in association with the header unit such that a second kit according to the invention comprises a source of propellant, a dispensing receptacle according to the invention and a second delivery device housing according to the invention.

The dispensing receptacle according to the invention comprises a receptacle containing a medicament unit in fluid tight engagement with a header unit wherein the header unit provides the receptacle with an inlet for propellant and an outlet wherein the outlet is spaced from the medicament to allow aerosolisation of the medicament in use and wherein the header unit has a propellant entry connector in fluid communication with the inlet for propellant.

The second delivery device housing according to the invention has a first open-ended compartment which is adapted to receive a source of propellant and a clip which is adapted to receive a dispensing receptacle according to the invention wherein the clip has a propellant connector associated with it which exit connector is arranged to engage with the entry connector of the dispensing receptacle.

A first kit according to the invention preferably comprises a plurality of receptacles. Optionally in the first kit, the receptacle and source of propellant may be provided in the form of combined supply for the first delivery device housing such that the receptacle and source of propellant are linked for combined insertion into the housing.

The receptacle containing the medicament can be any suitable packaging container, for example, a glass or plastic vial or a blister pack. Typically the opening of the receptacle is sealed to preserve sterility of the powder and avoid water adsorption. After removal of the seal the receptacle may then inserted into the device according to the invention such that the opening of the receptacle is brought into a fluid-tight engagement with the housing, preferably via a gasket or sealing ring. The receptacle may be held in engagement with the housing by a screw or twist connection. Alternatively a clamp on the housing or a closure (such as an end cap) for the other end of the compartment may be provided to support the receptacle and to press the opening of the receptacle against the housing or gasket, if present. The receptacle may contain a single dose of powder for one-time use, or sufficient powder for several doses. The medicament is preferably in the form of a respirable powder. More preferably the medicament is in the form of a powder having a mass median aerodynamic diameter (MMAD) measured by laser diffraction of less than 20 μm, preferably less than 10 μm, more preferably less than 5 μm, most preferably from 1 μm to 5 μm.

Where the receptacle is a vial, the spaced arrangement of the outlet is provided by the vial. This is because there is typically empty space between the contents of the vial and its opening. For a 10 ml vial, the volume of the contents is usually from 0.5 to 2 ml, leaving an empty volume of 8 to 9.5 ml. If the outlet of the device of the invention is formed in the header unit, this empty volume has been found to be sufficient to provide the spaced arrangement between the medicament and the outlet for certain medicaments, particularly pumactant.

Where a blister pack is used as the receptacle, the device preferably comprises an open-ended compartment for receiving the blister pack. The volume of the open-ended compartment preferably provides the spaced arrangement for the outlet. This is because in a blister pack there is usually insufficient volume between the opening of the blister pack and the medicament for this volume to be used as the volume for the spaced arrangement of the outlet.

This volume of the spaced arrangement of the outlet is preferably chosen according to the amount of medicament to be aerosolised and its degree of cohesion. It is preferably not so small that the medicament cannot be aerosolised. Also it is preferably not so large that the aerosol of the medicament is dissipated and destabilises.

The source of propellant is generally arranged in fluid-tight engagement with the propellant pathway by a screw, twist or push connection. Where the source of propellant is a gas canister, it is preferably a replaceable canister with a metering valve having an extended valve stem which is pressed to discharge gas.

The device is preferably arranged such that in use the valve is above the canister. This is advantageous because a patient can then use a thumb to activate the canister by pressing on its base. When using an MDI, the patient is instructed to use a finger to activate it. As substantial pressure can be required to activate a metered valve, this arrangement can lead to compliance problems which the present invention overcomes.

The device of the invention can be used to administer any medicament suitable for administration by inhalation such as a SAPL (surface active phospholipid) composition, such as pumactant, a bronchodilator or a steroid.

The propellant used in the present invention is preferably carbon dioxide, nitrogen, air, or a halocarbon (e.g. a fluorocarbon such as HFA-134a or HFC-227).

The invention is illustrated by way of example by the Figures of the accompanying drawings in which:

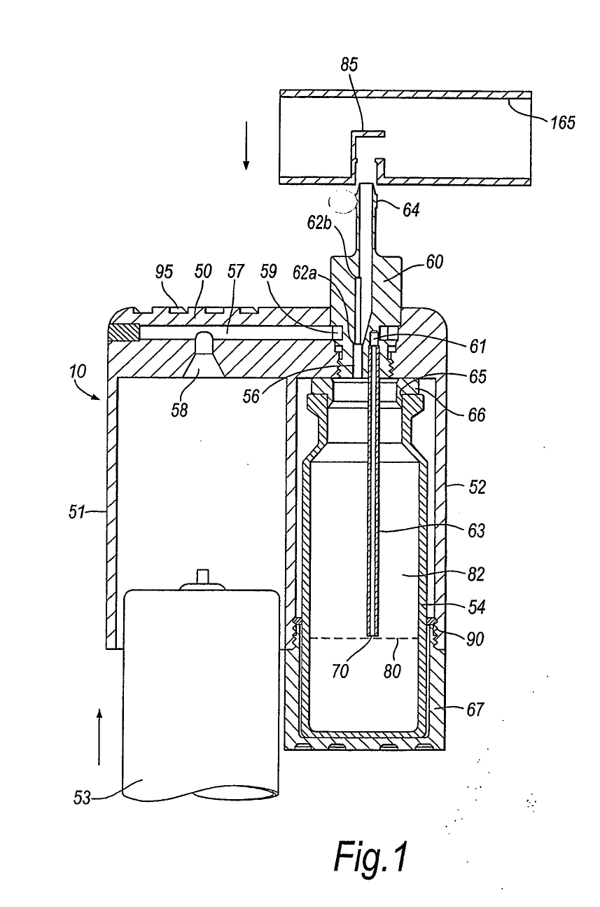

FIG. 1 is a cross-sectional view of a first embodiment of a device according to the invention;

FIG. 1a is a plan view of the device shown in FIG. 1;

FIG. 1b is a perspective view of a part of the device shown in FIG. 1;

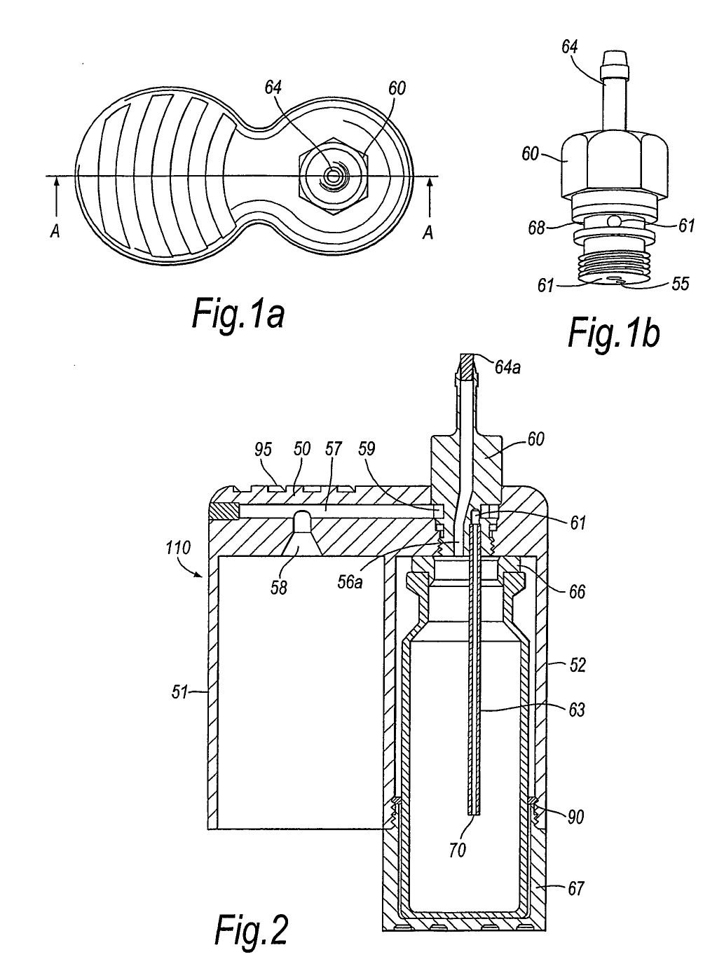

FIG. 2 is a cross-sectional view of a second embodiment of a device according to the invention;

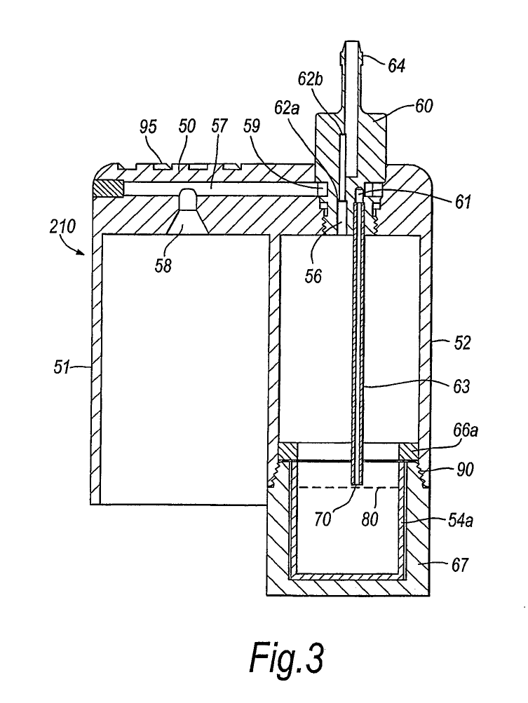

FIG. 3 is a cross-sectional view of a third embodiment of a device according to the invention;

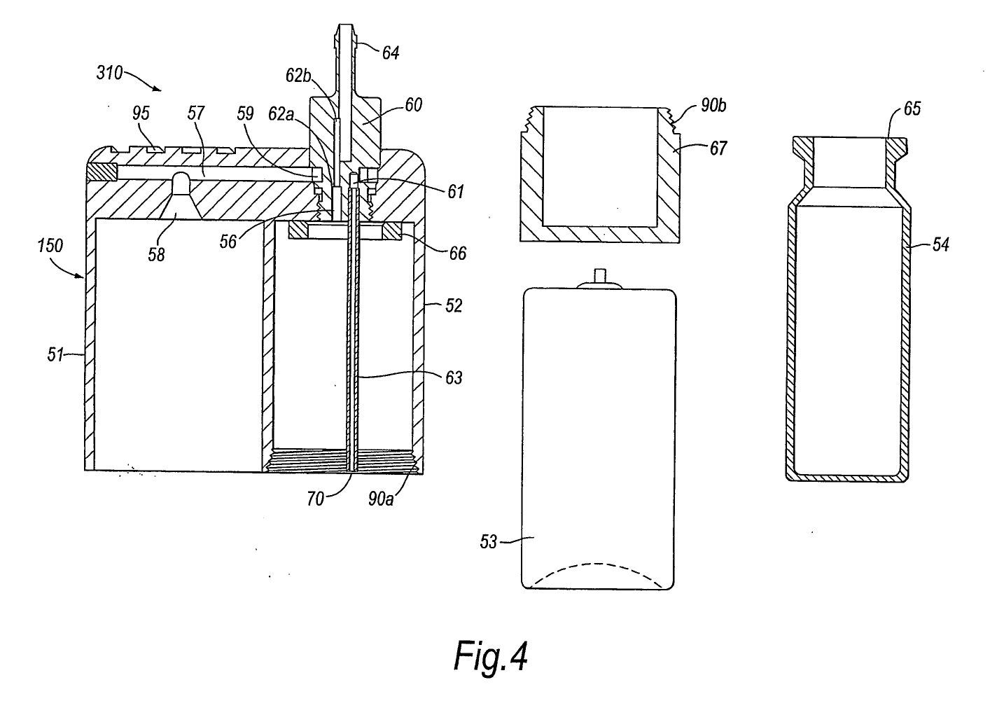

FIG. 4 is a cross-sectional view of a first embodiment of a kit according to the invention;

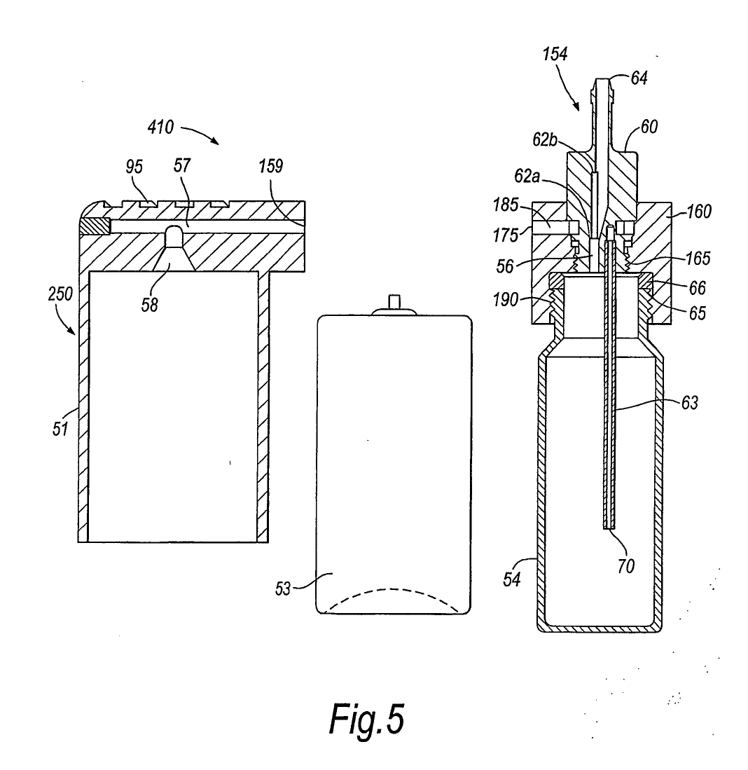

FIG. 5 is a cross-sectional view of a second embodiment of a kit according to the invention;

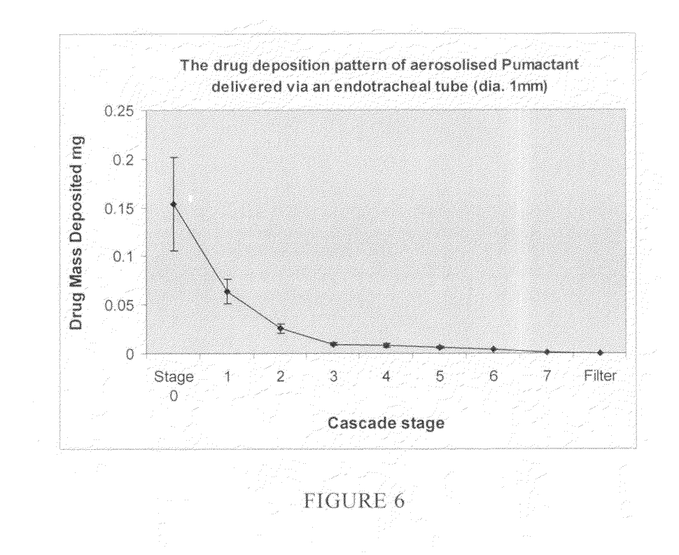

FIG. 6 shows a graph illustrating the data obtained from an in-vitro assessment of Pumactant aerosolised and delivered by a device according to the invention using a 1.5 m long 1 mm diameter endotracheal tube;

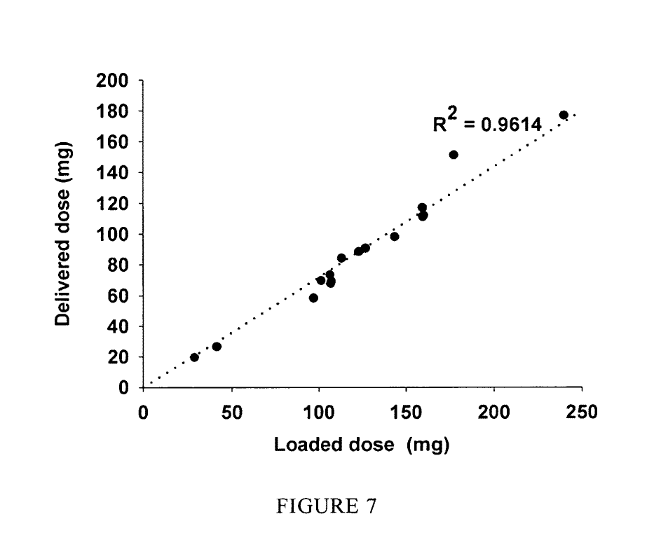

FIG. 7 shows the relationship between loaded dose and delivered dose in the procedure of Example 2; and

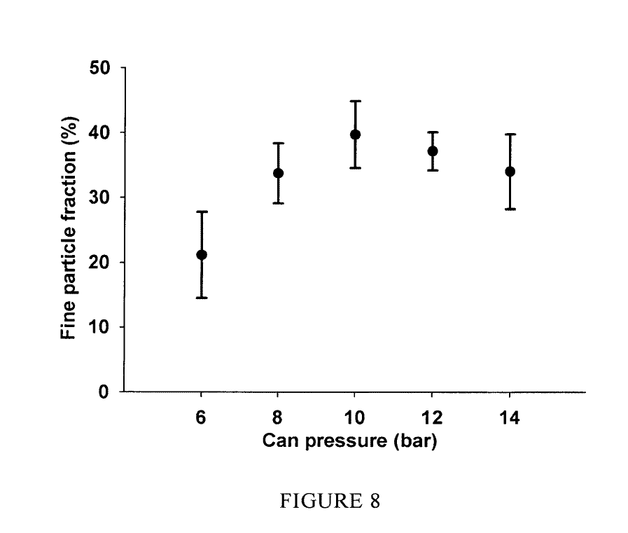

FIG. 8 charts fine particle fractions as a function of canister pressure in the procedure of Example 2.

A first embodiment of a dispenser device 10 of this invention is shown in FIGS. 1, 1a and 1b of the accompanying drawings. This embodiment has a housing 50 in the form of two open-ended cylinders 51,52 mounted side by side and forming respective chambers to hold a canister of pressurised propellant 53 (shown in part) and a receptacle 54 of medicament in powder form. The upper surface 95 of the housing 50 is moulded to provide a ridge surface to aid a patient's grip on the device.

A propellant pathway 57 is provided through the housing 50. The propellant pathway 57 links a propellant inlet fitting 58 for propellant formed at the top end of cylinder 51 and an aperture 59 formed in the end portion 56. Aperture 59 has a smaller cross-section than that of the propellant pathway 57 such that it provides a propellant pathway choke to decelerate fluid flow through the propellant pathway 57. In an alternative embodiment, the propellant pathway choke is in the form of a baffle.

The aperture 59 is adjacent to a screw-in header unit 60 seen in more detail in FIG. 1b. The header unit 60 has a circumferential groove 68. The housing 50 and header unit 60 are arranged such that the passage way 57 meets the circumferential groove 68. The groove 68 provides a further propellant pathway choke which is in the form of a baffle. The header unit 60 has an inlet pathway 61 which exits the base of the header unit 60. The direction of the inlet pathway 61 is at an angle of approximately 90 degrees to the propellant pathway 57. Thus where groove 68 and the inlet pathway 61 meet, a further propellant pathway choke is provided in the form of a baffle.

In an alternative embodiment, the header unit 60 is integrally moulded with the housing 50 such that the features of the header unit 60 are provided by the housing itself.

An inlet tube 63 is inserted into the pathway 61 in the base of the header unit 60 and extends into the interior of the cylinder 52. Thus the inlet tube 63 extends into the receptacle 54. An outlet 55 is also formed as a hole in the base of the header unit 60. Outlet 55 does not extend into the receptacle 54. Outlet 55 is spaced from the opening 65 of the receptacle 54 by a gasket 66 which seals the receptacle. In an alternative embodiment, outlet 55 is substantially flush with the opening 65 of receptacle 54. Outlet 55 is in fluid communication with outlet pathway 56 which extends to an outlet port 64 on the outer surface of the header unit 60.

Outlet pathway 56 is provided with a constriction 62a where the cross-section of outlet pathway 56 is reduced. Outlet pathway 56 is also provided with a baffle 62b. Constriction 62a and baffle 62b are arranged to decelerate fluid flow through outlet pathway 56.

The base of the header unit 60 is provided with a gasket 66 which provides a fluid-tight seal between the header unit 60 and receptacle 54. The receptacle 54 is held tightly against gasket 66 because the open-end of cylinder 52 is sealed by screw-threaded end cap 67.

The propellant canister 53 is provided as a replaceable unit, and most suitably contains a compressed gas as propellant, such as carbon dioxide, nitrogen or air. However other conventional propellants, such as a low boiling liquid, preferably a fluorocarbon such as HFA-134a or HFC-227, under sufficient pressure to maintain the propellant liquid at normal room temperature, may also be used. The propellant canister 53 is a conventional unit which has a metering valve with a protruding valve stem, which when depressed releases propellant through a passage way in the valve stem. In use of the device, the canister 53 is inserted into the cylinder 51 so that the valve stem is located in gas inlet fitting 58. The fitting 58 is dimensioned so that the valve stem is a press fit in the fitting 58 and so holds the canister 53 in the interior of the cylinder 51.

The receptacle 54 containing medicament, is typically supplied as a sealed unit. Receptacle 54 has an opening 65 which before use is sealed to protect the powder contents. After stripping the seal, the receptacle 54 is introduced into the interior of the cylinder 52, so that the opening 65 is forced against a resilient gasket 66 and the delivery tube 63 enters into the receptacle 54. The open end of the cylinder 52 is closed with an end cap 67 which engages with the cylinder 52 by a mutual screw-thread 90. The end cap 67 provides the means by which the receptacle 54 is maintained in position with the opening 65 sealingly engaged with the gasket 66.

An alternative arrangement to FIG. 1 is shown in FIG. 3. This device 210 is the third embodiment of the device according to the invention. Like reference numerals are used to represent like features of the first embodiment. In this embodiment, receptacle 54a is smaller than the receptacle 54 shown in FIG. 1. This receptacle 54a is in the form of a blister pack. Here there is a much smaller gap between the opening 65 of the receptacle 54 and the medicament level 80. A gasket 66a is provided adjacent to screw-thread 90. This is in order that in use, a blister pack 54a can be placed into end cap 67 which is then used to close cylinder 52. The mouth 65 of blister pack 54a then engages with gasket 66a which holds the blister pack in place. The outlet tip is directed against the medicament in the blister pack 54a. The device 210 works in the same way as the device 10 according to the first embodiment of the invention.

To use the device, the user pushes the end of the gas canister 53 into the interior of the cylinder 51. As the valve stem of the canister remains secured in the passage 58, the inward movement of the canister effectively depresses the valve stem, and releases propellant through the valve stem into the passageway 57. The propellant proceeds through aperture 59, circumferential groove 68, inlet passage 61 and into the receptacle 54 via delivery tube 63. The delivery tube 63 is dimensioned so that its outlet tip 70 is directed at or dipping into the powder contents of the receptacle 54, so that the propellant is directed against the powder. (To avoid damage when the closure 67 is removed and no receptacle 54 is loaded, the tube 63 is dimensioned so that the tip 70 lies within the cylinder 52). As a result, the propellant fluidises the powder and forms a respirable aerosol in the volume 82 between the level of the medicament 80 and the outlet 55. The aerosol exits the receptacle 54 via the outlet 55 and the outlet passage 56. On its way through the outlet passage, the aerosol is decelerated by constriction 62a and baffle 62b.

The outlet port 64 may be formed as, or exit into, a mouthpiece 165 or a shaped end piece which is a comfortable shape to place in the mouth, nose or other body orifice of a patient. The mouthpiece 165 shown has a baffle 85. Alternatively the outlet 64 may be extended to form, or connect to, a respiration tube, e.g. a tracheal tube (not shown).

A second embodiment of a dispenser device 110 according to the invention is shown in FIG. 2. Like reference numerals are used to represent like features of the first embodiment. The device 110 differs from device 10 in that outlet pathway 56a lacks the constriction 62a and baffle 62b of the first embodiment. The device 110 also differs in that outlet port is sealed with removable seal 64a. The device 110 works in the same way as device 10 except that it is suitable for optimisation to generate a stable aerosol or standing cloud on activation.

As an alternative in device 110, removable seal 64a is replaced by a normal outlet port 64.

A first embodiment of a kit 310 according to the invention is shown in FIG. 4. Kit 310 has a device housing 150, an end cap 67, a source of propellant 53 and a receptacle 54. Like reference numerals are used to represent like features of the first embodiment.

A second embodiment of a kit 410 according to the invention is shown in FIG. 5. Kit 410 has a device housing 250, a source of propellant 53 and a dispensing receptacle 154. Like reference numerals are used to represent like features of the first embodiment.

Device housing 250 has a propellant exit connector 159 which is provided with a constriction to act as a propellant pathway choke. Device housing 250 also has a clip (not shown) for engaging dispensing receptacle 154.

Dispensing receptacle 154 has a receptacle connector 160, header unit 60 and receptacle 54. Receptacle connector 160 joins header unit 60 to receptacle 54. Header unit 60 engages with the receptacle connector 160 by screw fitting 165 and receptacle 54 engages with the receptacle connector 160 by screw fitting 190. Receptacle connector 160 has a propellant entry connector 175 which is in fluid communication with a propellant pathway 185 which leads to circumferential groove 68 on the header unit 60.

To use the kit according to the second embodiment, the dispensing receptacle 154 is clipped onto the device housing 250 such that the propellant exit connector 159 of the device housing 250 engages with the propellant entry connector 175 of the receptacle connector 160. The assembled kit then functions in the same way as the device 10 according to the first embodiment of the invention.

The efficacy of the device according to the invention is illustrated in the following Examples:

### EXAMPLE 1

A device according to the invention has been successfully used in experimental veterinary treatment of respiratory disorders in horses using pumactant, as detailed below.

Horses are susceptible to a plethora of respiratory complaints. Heaves is the equine equivalent of asthma and both diseases share similar aetiology and pathology. The disease, in the equid, has been shown to proceed via a Th2 cytokine driven mechanism (Lavoie, J-P., Maghni, K., Desnoyers, M., Taha, R., Martin, J. G., and Hamid Q. A.(2001) Neutrophilic airway inflammation in horses with heaves is characterised by a Th2 cytokine profile. Am. J. Respir. Crit. Care. Med 164 1410-1413). They, like their human counterparts, have poor compliance and a massive lung surface area estimated to be in the region of 1000 m².

The aim of the study was to investigate the use and approach to delivery of a thermally labile, hygroscopic and dry surfactant, ensuring an acceptable physicochemical character. The surfactant used was pumactant, (formerly known as ALEC), which is a mixture of two phospholipids: DPPC and PG in a ratio of 7 parts:3 parts DPPC:PG. This specific ratio of phospholipids has a low phase transition temperature (approximately 32° C.) which it is believed facilitates rapid spreading at body temperature when in contact with an air/water interface. It is also highly rich in DPPC which mimics the high percentage of endogenous DPPC in vivo.

It was used as a dry powder because in a previous human (allergic asthma) study (Babu, K. S., Woodcock, D. A., Smith, S. E., Heminsley, A. M., Little, L., Staniforth, J. N., Holgate, S. T., and Conway, J. H. Pumactant abolishes early asthmatic response in patients with allergic asthma, Presentation given at the American Thoracic Society, Atlanta, USA (2002)), the preparation had been delivered as a dry powder and produced excellent clinical results. Currently, surfactants are delivered as aqueous based preparations, however it is has been demonstrated that surface activity is reduced when the active is delivered as an aqueous suspension. Indeed, delivery of aqueous preparations is counterintuitive in certain disease states: RDS.

Pumactant is physically unstable even at conditions of low relative humidity (approximately 30%), and it can undergo morphological changes, which may affect particle size. Careful attention must therefore be applied to storage and delivery of the surfactant.

The device according to the present invention was used for delivery of pumactant because it has the following advantageous delivery characteristics:

The use of a particulate free and low moisture gas source
Capable of aerosolising and de-aggregating large particles
Adaptation to equine anatomy and physiology
Ease of use for clinician/veterinarian

The pumactant was administered by utilising an endotracheal tube, bypassing the nasal anatomy, delivering the material to each bronchus; this arrangement, obviously, would also omit patient compliance issues.

The use of an equine model, as previously described, facilitated the delivery of a mass of powder not conventionally delivered to the respiratory tract. The device and mode of delivery is erstwhile described, but what is not apparent is the particle size distribution of the material used. Since it was manufactured as freeze dried powder the particle size distribution does not conform to a conventional respiratory particle size distribution. In fact, the MMAD (mass median aerodynamic diameter) as evaluated by laser diffraction was approximately 10 microns with a distribution that ranged from approximately 1 to 180 microns. This was initially a concern. Current practice delivers particles in a 2-5 MMAD micron range and, whilst direct delivery to each bronchus removed some proximal deposition, it had not been established the extent to which a large particle would penetrate.

The deposition was measured in vitro using an Andersen cascade impactor. The results are given in FIG. 6.

The following results were obtained. (The initial baseline assessment from tracheal washings are given in Table 1.)

TABLE 1
BASELINE ASSESSMENT OF SUBJECT
PRIOR TO STUDY START

| Macroscopic appearance | | Microscopic appearance | |
|---|---|---|---|
| Mucus | +++ | Neutrophils | +++ |
| Cloudy | trace | Deg Neutrophils | + |
| Blood | + | Macrophages | + |
| | | Siderophages | + |
| | | Epithelium | ++ |

General Inflammation score (0-12) 7
Wherein the following scoring severity was used:
−−− = non detected,
+ = mild,
++++ = severe

The results obtained during the term of the study are illustrated in Table 2.

TABLE 2
TRACHEAL WASH DATA COLLECTED DURING THE
TERM OF THE STUDY

| | Nucleated | | | | |
|---|---|---|---|---|---|
| | Cells/1 | Neutro- | Mono- | | Epi- |
| Date | Cell Type | phils | nuclear | Eosionphils | thelium |
| 19 Jan. 2002 | 0.3 × 10⁹ | −−−− | −−−− | −−−− | −−−− |
| (24 hours | | | | | |
| post | | | | | |
| treatment) | | | | | |
| 26 Jan. 2002 | 1.2 × 10⁹ | ++ | ++ | + | + |
| (Pre | | | | | |
| treatment) | | | | | |
| 26 Jan. 2002 | 0.8 × 10⁸ | + | ++ | −−− | ++ |
| 22 Feb. 2002 | HIGH | 28% | 24% | −−− | 48% |
| centrifuged | | | | | |
| deposit | | | | | |
| smear cell | | | | | |
| density | | | | | |
| (Pre | | | | | |
| treatment) | | | | | |
| 25 Feb. 2002 | LOW | 32% | 27% | 3% | 38% |
| centrifuged | | | | | |
| deposit | | | | | |
| smear cell | | | | | |
| density | | | | | |
| (Post | | | | | |
| treatment) | | | | | |

This Example shows the use of the device according to the invention in administering phospholipids in the treatment of equine respiratory disorders: Heaves in this instance. Primarily, the treatment is hypothesised to ‘work above the line’: to form a barrier over the epithelial surface it contacts with. The results from Table 2 indicate a reduction in epithelial shedding. When the epithelium is denuded or missing, the tissues below are exposed to insult, allowing the cascade of subsequent inflammatory mechanisms to proceed.

### EXAMPLE 2

The performance of an inhaler as shown in FIG. 1 was investigated using pumactant as a model drug. In particular, the influence of loaded dose on dry powder delivery and can pressure on aerosolisation efficiency was investigated.

Reported clinical studies required a dosage regime of 4×100 mg, 8 hours and 30 mins prior to an allergen challenge [Babu, K S. et al, ibid]. Such high doses were well tolerated and early asthmatic response was abolished in all cases. However, due to pumactant's similarity to endogenous surfactant (e.g. low transition temperature and high moisture affinity), the energy required to aerosolise the powder was not achievable using conventional means.

### Physical Characterisation of Pumactant

Prior to in vitro testing, the micronised pumactant was first characterised for particle morphology, size distribution, moisture sorption and crystal structure.

The particle morphology of the micronised pumactant was investigated using scanning electron microscopy (SEM) (Jeol 6310: Jeol, Japan). Samples were mounted on carbon sticky tabs prior to analysis and gold coated (Edwards Sputter Coater, UK). Analysis of the data suggests discrete particulates with diameters less than 5 μm. Furthermore, the micronised particles appeared heavily agglomerated.

The particle-size distribution of the micronised pumactant was determined by laser light scattering (Mastersizer X, Malvern, UK), using a 100 mm lens and small volume stirring circulation cell. The micronised powder was dispersed in cyclohexane and ultrasonicated for 5 minutes prior to analysis (determined sufficient to fully de-aggregate the powder).

The median volumetric diameter (d0.5) for micronised pumactant was 1.49±0.12 μm (n=3). Furthermore, the 10^{th} and 90^{th} percentile particle diameters were 0.81 μm±0.06 μm and 2.92 μm±0.31 μm, respectively suggesting the micronised drug to be of suitable size for inhalation therapy [Pritchard, J. N. 2001. The influence of lung deposition on clinical response. J. Aerosol Med. 14:S19-S26]. The particle size distribution appeared to be in good agreement with observations made by SEM.

In general, physical characterisation of the pumactant suggests the potential of aerosolisation would be relatively low. The powder has a micron size (<5 μm) and thus high surface area to mass ratio (cohesion), Furthermore, the material appeared heavily agglomerated, contained significant quantities of water and was predominately amorphous.

Moisture sorption profiles of the micronised Pumactant was conducted using dynamic vapour sorption (DVS) (DVS-1 Surface Measurement Systems, London, UK). Approximately 12 mg of powder was weighed into the sample pan of the DVS and subjected to a 0-90% relative humidity (RH) cycle (10% increments). Equilibration at each humidity was determined by a dm/dt of 0.0002%.min⁻¹.

The test results showed that initial water uptake at each specific humidity was very rapid (<30 mins) before stabilisation. In general, an increase in mass of 14% was observed as humidity was increased from 0% RH to 90% RH. At 45% RH the percentage moisture content was approximately 6.2%. The subsequent in vitro studies were conducted at 45% RH (25° C.), and thus it would be reasonable to assume pumactant would be partially hydrated material.

Diffraction patterns for the micronised pumactant were obtained using X-ray powder diffraction (XRPD) using components and methods described elsewhere [Tobyn, M. J., McCarthy, G. P., Staniforth, J. N., Edge, S. 1998. Physicochemical comparison between microcrystalline cellulose and solidified microcrystalline cellulose. Int. J. Pharrn. 169:183-1 94].

Analysis of the XRPD diffractograph suggests a predominately amorphous material. Such observations are expected however, since the final two stages of pumactant production involves vacuum drying from an ethanol solution followed by cryo-micronisation. It is interesting to note however, that a broad peak was observed at 21° 2Θ, suggesting the presence of small semi-crystalline, or crystallite material in the powder.

### Dispenser Device

The influence of loaded dose (20-250 mg) on delivery efficiency and can pressure (6-14 bar) on aerosolisation efficiency (120 mg dose) was investigated. Pressurised canisters were filled with N₂ (O₂ free) (BOC, Manchester, UK), using a hand held pressurised filling machine (Manual Lab Plant, Pamasol, Switzerland), to 6, 8, 10, 12 and 14 bar (1×10⁵ Pa) pressures. Filling pressures were checked against a calibrated pressure meter (Pamasol P700, Switzerland).

### Delivered Dose Studies

The influence of loaded dose (0-250 mg) on the delivered dose (aerosolisation of the powder bed) was investigated. Samples of pumactant were accurately weighed into pre-weighed sample vials, which were inserted into the device. Studies were conducted using 12 bar N₂ canisters. The device was actuated for a 10 second period into a fume hood. Delivered dose was calculated by mass difference. The device and actuator were cleaned using methanol and air-dried. All experiments were conducted at 45% RH and 25° C., and were randomised for loaded dose.

### Aerosolisation Efficiency Studies

The influence of can pressure on the aerosolisation efficiency of 120 mg pumactant doses was investigated using the Marple Miller impactor (USP Apparatus 2) (Copley Instruments Ltd, Nottingham, UK). The Marple Miller impactor has five collection stages (in the form of sample cups), which at 60 L·min⁻¹ produce 5 effective aerodynamic cut-off diameters; 10 μm, 5 μm, 2.5 μm, 1,25 μm and 0.625 μm. In addition, a throat and after-filter provide collection of particles >10 μm and <0.625 μm. A rotary-vein pump (Gast, Buckinghamshire, UK) generated a flow rate of 60 L·min⁻¹ through the impactor, which was calibrated using a flow meter.

Approximately 120 mg of pumactant was weighed into a pre-weighed sample vial, which was inserted into the device. The actuator mouthpiece was inserted into a specially constructed mouthpiece and tested using the Marple Miller impactor at 60 L·min⁻¹ for 10 seconds. A 3 second delay prior to pressurised can actuation was instigated to allow equilibration of the pump. Drug concentrations in the sample vial, device and Marple Miller stages were calculated by mass difference using a 5-figure Sartorius balance. Data were processed to produce delivered dose (DD) (ex device), fine particle dose (FPD) (mass in stage 2 to filter) and fine particle fraction (FPF) (FPD/DD×100). The FPD and FPF refer to deposited drug with an aerodynamic mass median diameter of less than <5 μm. The Marple Miller sample cups, filter stage throat and device were cleaned with methanol and air-dried between experiments.

As with the delivered dose studies, environmental conditions were 45% RH and 25° C. Experiments were randomised for can pressure.

### Pumactant Aerosolisation Efficiency

The efficiency of the device in delivering micronised pumactant was investigated. Initially the relationship between loaded dose and delivered dose (0-250 mg) was studied (12 bar canister pressure). Secondly, the aerosolisation efficiency of the micronised pumactant (i.e. particles that would potentially be respirable (<5 μm)) was investigated as a function of canister pressure (6-14 bar). In this case a 120 mg loaded dose was chosen for similarity to clinical trial doses reported previously.

### Delivered Dose Studies

The relationship between loaded and delivered dose is represented graphically in FIG. 7. In general, a linear relationship (R²=0.96) between loaded and delivered dose was observed (n=18). Device efficiency across all doses was 70.1%±6.3% (n=18). As expected, no correlation between loaded dose and device efficiency was found (Pearson analysis).

### Influence of Canister Pressure on Fine Particle Aerosolisation

The influence of can pressure on the aerosolisation efficiency of the PADD device, using a Marple Miller impactor, is summarised in the Table 3 and illustrated in FIG. 8.

TABLE 3
INFLUENCE OF CAN PRESSURE
ON AEROSOLISATION EFFICIENCY

| | | | Fine particle | Fine particle |
|---|---|---|---|---|
| Pressure | Loaded dose, | Delivered dose, | dose,² | fraction,³ |
| (bars¹) | (mg ± sd) | (mg ± sd) | (mg ± sd) | (% ± sd) |
| 6 | 120.7 ± 1.5 | 35.7 ± 8.8 | 7.5 ± 2.7 | 21.1 ± 6.6 |
| 8 | 118.4 ± 6.6 | 79.3 ± 10.1 | 27.0 ± 7.1 | 33.7 ± 4.6 |
| 10 | 116.8 ± 1.4 | 79.2 ± 7.7 | 31.4 ± 5.1 | 39.7 ± 5.2 |
| 12 | 121.0 ± 7.0 | 86.4 ± 2.8 | 32.1 ± 3.0 | 37.2 ± 3.0 |
| 14 | 120.4 ± 0.9 | 86.8 ± 6.5 | 29.3 ± 3.0 | 34.0 ± 5.8 |

¹1 bar = 1 × 10⁵ Pa,
²Deposited fraction collected from stage 2-filter (<5 μm),
³Percentage fraction below 5 μm

The mean loaded dose throughout the study was 119.5±4.1 mg. Statistical analysis (ANOVA, Fisher pair wise, p<0.05) indicated no significant variance between loaded doses and canister pressure studied.

Statistical analysis of delivered dose (ANOVA, p<0.05) indicated canister pressure had significant influence on powder bed fluidisation. However, Fisher's pair-wise analysis indicated this to only be the case between 6 and 8 bars (35.7 mg±8.8 mg at 6 bar to 79.3 mg±10.1 mg at 8 bar). Thus, it is reasonable to suggest that the device could be successfully used between 8 and 14 bars.

Although delivered dose is a good estimation of the powder bed fluidisation efficiency, it is not indicative of the aerosolisation efficiency of the system (that is to say, the efficiency of the system in de-agglomerating the micronised powder agglomerates). The fine particle dose therefore is used to describe the potential dose that would be received in the lower respiratory tract (lower bronchiole) [Pritchard supra].

Previous investigations using micronised pumactant (˜50 mg) and a commercial dry powder inhaler (Cyclohaler®, Novartis, Surrey, UK), showed comparable delivered dose values to the present device, but resulted in no FPD [Young, P. M., Thompson, J., Price, R., Woodcock, D., Davies, K. 2003. The use of a novel hand held device to deliver high respirable fractions of high dose dry powder active agents to the lung. J. Aerosol Med. 16:192]. Such observations suggested the energy of the Cyclohaler® was not sufficient to de-agglomerate the powder once entrained in an air stream. In comparison, the mean FPD using the present device and 6 bar canister was 7.5±2.7 mg (n=3). This rose significantly (Fisher's pair-wise, p<0.05) to 27.0 mg±7.1 mg at 8 bar (n=3). Further increases in canister pressure did not result in significant changes in FPD. However, it is interesting to note a decrease in the standard deviation was observed as pressure was increased (with a FPD of 29.3 mg±3.0 mg being observed at 14 bar (n=3)).

Comparison of the FPF indicated similar findings to the FPD, with a significant increase (Fisher pair-wise, p<0.05) in FPF between 6 and 8 bar canister pressures (21.1 mg±6.6 mg and 33.7 mg±4.6 mg at 6 and 8 bars, respectively). However, the relative difference between 6 and 8 bar FPF values when compared with FPD was less. Such observations are most likely attributed to the relative differences in delivered doses between the two pressures. Again, no significant difference (ANOVA, Fisher pair-wise, p<0.05) in the FPF for tests conducted between 8-14 bar pressurised canisters were observed. A mean FPF of 36.1 mg±4.8 mg was observed across the range: 8-14 bar.

Initial studies using the pressurised aerosol dry-powder delivery device according to the invention show that the aerosolisation of micronised pumactant is possible over the range 20-250 mg. Furthermore, in vitro studies of 120 mg loaded doses indicated fine particle fractions of >30 weight % (˜30 mg FPD) when delivered using 8-14 bar aerosolisation pressures. Although previous studies have demonstrated the delivery of high dose medicaments is possible, the combination of active device design and carrier free formulation enables high energy powder aerosolisation while circumventing issues that may arise with the use of high dose excipients.

1.-48. (canceled)

49. A delivery device for a medicament, comprising:
a housing,
a receptacle holding a medicament in the form of a powder, and
a source of propellant in the form of a canister of gas, wherein the housing provides an inlet and an outlet for the receptacle, wherein the inlet is in fluid communication with the source of propellant, the inlet is directed against the medicament, and the outlet is spaced from the medicament to allow aerosolization of the medicament, wherein the outlet is in fluid communication with an outlet pathway which connects to the exterior of the device, wherein the outlet pathway is provided with one or more outlet pathway chokes for decelerating the aerosol of the medicament.

50. The device according to claim 49, wherein the receptacle is removable from the housing.

51. The device according to claim 49, wherein the source of propellant is removable from the housing.

52. The device according to claim 49, wherein the fluid communication between the inlet and the source of propellant is provided by a propellant pathway, the propellant pathway having at least one propellant pathway choke means to decelerate the propellant.

53. The device according to claim 49, wherein the inlet has an end which is directed against the medicament, and the end is in the form of a flared tube or of a shower-head.

54. The device according to claim 49, wherein the inlet is in the form of an inlet tube that may be provided with one or more perforations and that may be extendible into the receptacle.

55. The device according to claim 49, wherein the receptacle has a bottom containing the medicament and a top which connects to the housing, and the outlet is arranged to open into the receptacle at the top of the receptacle.

56. The device according to claim 55, wherein the outlet opens into the receptacle at one end of the outlet, and the one end of the outlet is substantially flush with the top of the receptacle.

57. The device according to claim 49, wherein the outlet does not extend into the receptacle, a mouthpiece attachable to the outlet, the outlet adapted to be provided with a tube for engaging with a breathing tube for a patient using a respirator or a mouthpiece attached thereto.

58. The device according to claim 49, wherein the outlet is formed as a hole in the housing which is in fluid communication with an outlet pathway in the housing which connects to the exterior of the housing.

59. The device according to claim 49, wherein the canister of gas has a valve and wherein the device is arranged such that in use the valve is above the canister.

60. The device according to claim 49, wherein the receptacle is in the form of an open-ended compartment and an optionally removable blister pack.

61. A housing for a delivery device for a medicament, comprising:
a first and a second open-ended compartment, wherein the first compartment is adapted to receive a source of propellant in the form of a canister of gas and the second compartment is adapted to receive a receptacle containing a medicament in powder form, wherein the second compartment provides an inlet for propellant in fluid communication with the first compartment and an outlet, wherein the outlet, in use, is spaced from the medicament to allow aerosolization of the medicament, wherein the outlet is in fluid communication with an outlet pathway which connects to the exterior of the device, wherein the outlet pathway is provided with one or more outlet pathway chokes for decelerating the aerosol of the medicament.

62. A kit comprising a canister of propellant, at least one receptacle containing a medicament in powder form and the delivery device housing according to claim 61.

63. The kit according to claim 61, wherein the receptacle and the propellant are provided in the form of combined supply for the delivery device housing such that the receptacle and the propellant are linked for combined insertion into the housing.

64. A dispensing receptacle, comprising:
a receptacle unit containing a medicament in powder form, which receptacle unit is in fluid tight engagement with a header unit, wherein the header unit provides the receptacle unit with an inlet for a propellant and an outlet, wherein the outlet is spaced from the medicament to allow aerosolization of the medicament, and wherein the header unit has a propellant entry connector in fluid communication with the inlet for the propellant, wherein the outlet is in fluid communication with an outlet pathway which connects to the exterior of the device, wherein the outlet pathway is provided with one or more outlet pathway chokes for decelerating the aerosol of the medicament.

65. The receptacle according to claim 64, wherein the inlet has an end which is directed against the medicament, and the end is in the form of an inlet tube, a flared tube or a shower-head.

66. The receptacle according to claim 64, wherein the outlet is in fluid communication with an outlet pathway which connects to the exterior of the header unit, wherein the outlet pathway is provided with one or more outlet pathway choke means for decelerating the aerosol of the medicament.

67. The receptacle of claim 64, wherein the receptacle is adapted to be received by a clip of a housing for a delivery device for a medicament that includes a first open-ended compartment which is adapted to receive a source of propellant, wherein the clip is associated with a propellant exit connector in fluid communication with the first compartment, and the exit connector is arranged to engage with an entry connector of the dispensing receptacle.

68. The receptacle of claim 67, wherein the fluid communication between the propellant exit connector and the first compartment is provided by a propellant pathway which has at least one propellant pathway choke to decelerate the propellant.
